(19) United States
(12) Patent Application Publication
(10) Pub. No.: US 2013/0245380 A1
(43) Pub. Date: Sep. 19, 2013

(54) Hydro-Colonoscopy Combination System

(71) Applicant: Delmar Vogel, North Vancouver (CA)

(72) Inventor: Delmar Vogel, North Vancouver (CA)

(21) Appl. No.: 13/421,417

(22) Filed: Mar. 15, 2012

(51) Int. Cl. A61M 3/02; A61B 1/32

(52) U.S. Cl. 600/205

(57) ABSTRACT

The present invention provides a speculum for performing colonoscopy in combination with colon irrigation/cleansing, a compact system comprising a colon irrigation device and a colonoscopy device, and a method for performing colonoscopy along with colon irrigation/cleansing. The present invention also provides a disposable kit with the speculum and a washer cone. The speculum according to the present invention comprises a main tube having a proximal end and distal end, and an inlet tube and an outlet tube intersecting to the main tube. The speculum of the present invention allows carrying out colonoscopy screening immediately following the colon irrigation which saves time.

### STATEMENT REGARDING FEDERALLY FUNDED RESEARCH

This invention was not made by any agency of the United States Government nor under any contract with any agency of the United States Government.

### FIELD OF THE INVENTION

The present invention relates to a colonoscopy. Preferably, the present invention relates to a colonoscopy in combination with colon irrigation or cleansing.

### BACKGROUND ART OF THE INVENTION

Colonoscopy means a colon screening, which is an endoscopic examination of the large bowel and the distal part of the small bowel with a CCD camera or a fiber optic camera adapted on a flexible tube, which is hereinafter referred to as Colonoscope or Scope. The scope is inserted through the anus for colonoscopy. General conditions that call for colonoscopies include gastrointestinal hemorrhage, unexplained changes in bowel habit, an unexplained drop in hematocrit (one sign of anemia) and suspicion of malignancy. Colonoscopies are often used to diagnose colon cancer, but are also frequently used to diagnose inflammatory bowel disease. Also, patients with a family history of colon cancer are often first screened during their teenage years.

To perform colonoscopy properly, the colon must be cleaned and free of solid matters. The cleansing of colon is known as a bowel preparation. Accordingly, as per the presently available techniques, the patient is required to follow a low fiber or clear-liquid only diet for one to three days. The day before the colonoscopy, the patient is given a laxative preparation and large quantities of fluid. Since the goal of the preparation is to clear the colon of solid matters, the patient further requires to plan on spending the day at home in comfortable surroundings with ready access to toilet facilities. Further, use of laxatives may have side effects. Alternatively, the bowel cleaning is performed using a solution of polyethylene glycol and electrolytes one day before the colonoscopy. This form of bowel cleaning may be suggested some time before the colon screening but at least one hour before the colonoscopy. Generally, this procedure involves both a pill-form laxative and a bowel cleaning preparation of polyethylene glycol powder dissolved into any clear liquid which may have adverse side effects.

Presently, the colonoscope is inserted into the colon and advanced along the colon using strong manual pushing force that can result in injury or colon perforation. Also, manual pushing results in a high level of pain that requires anesthesia. The use of anesthetics requires extra post treatment recovery time.

Further, incomplete or failed colonoscopies can happen due to not sufficiently cleaned colons resulting in the need to reschedule patients for a new appointment and repeat the entire colon screening including the full bowel preparation procedure.

Further, the presently available process consumes more time for bowel preparation and to carry out the colonoscopy.

### SUMMARY OF THE INVENTION

Accordingly, the present invention provides a device and method to carry out colonoscopy in combination with colon irrigation or cleansing as a means of bowel preparation.

In the first embodiment, the present invention provides a rectal speculum for carrying out colonoscopy in combination with colon irrigation or cleansing. The said speculum comprises a main tube having a distal end for insertion into a patient's rectum and a proximal end for inserting a colonoscope, an inlet tube for directing fluids into the said main tube, an outlet tube for connecting to a waste discharge hose, and a cap for closing the proximal end of the main tube for carrying out irrigation. The inlet tube and the outlet tube intersect the main tube of the speculum near the proximal end of the main tube.

According to the present invention, the inlet tube has a diameter less than the diameter of main tube and preferably intersects the main tube at an obtuse angle with respect to the distal end of the main tube.

According to the present invention, the outlet tube has a diameter substantially equal to the diameter of the said main tube and preferably intersects the said main tube at an obtuse angle with respect to the distal end.

According to the present invention, the inlet tube and the outlet tube are intersecting preferably adjacent to the main tube. Alternatively, the inlet tube and the outlet tube can be intersecting to the main tube away from each other.

According to the present invention, the inlet tube and the outlet tube are intersecting preferably at the same distance from the distal end of main tube. Alternatively, the inlet tube and the outlet tube can intersect at different distance from the distal end of the main tube.

In second embodiment, the present invention provides a rectal speculum kit for performing colonoscopy in combination with colon irrigation or cleansing, comprising a rectal speculum having an inlet tube and an outlet tube intersecting to a main tube, an obturator removably positioned in the main tube for inserting from a proximal end for facilitating insertion of speculum in a patient's rectum, a cap for closing the proximal end of the main tube for carrying irrigation and a penetration cap with a seal skirt for inserting a colonoscope from the proximal end of the main tube.

In the third embodiment, the present invention provides a system for carrying out colonoscopy in combination with colon irrigation/cleansing, comprising: a colon irrigation equipment for carrying out irrigation of the colon; a colonoscopy equipment for carrying out colonoscopy including a colonoscope and a buoyancy cuff to adapt at the tip of the colonoscope, a rectal speculum, said speculum having an inlet tube and an outlet tube intersecting to a main tube, a cap for closing a proximal end of the main tube for carrying out irrigation/cleansing, an obturator removably positioned in the main tube for facilitating insertion of speculum in a patient's rectum, and a penetration cap with a seal skirt for inserting a colonoscope from the proximal end of the main tube.

The present invention in third embodiment provides a method for carrying out colonoscopy in combination with colon irrigation/cleansing, Accordingly, a fluid hose of a colon irrigation device is fitted over the inlet tube for receiving irrigation fluid and waste removal hose to the outlet tube of the speculum. After fitting the hoses, the obturator is inserted from proximal end of the main tube and then the speculum is inserted into a rectum of the patient with slight twisting at a predetermined depth. After insertion, the obturator is removed from the speculum and colon irrigation/cleansing is carried out with repeated fill and empty cycles after closing the proximal end of the main tube. After a predetermined last empty cycle of irrigation, the cap is removed from the proximal end of the main tube of the speculum and colonoscopy is carried out by filling the colon slowly with the irrigation fluid and advancing the colonoscope up to secum of the colon; and emptying the colon. To carry out the colonoscopy, a tip of the colonoscope through the penetration cap and the seal skirt and the buoyancy cuff is attached to the tip of colonoscope and then fitting the penetration cap on the proximal end of the speculum. Advantageously, the speculum is inserted through a washer cone made of soft gel material adapted for snugly fitting of the speculum over the rectal area of the patient and providing comfort to the patient and prevents leaking of waste fluid material between speculum and anal wall.

In forth embodiment, the present invention provides a rectal speculum comprising a main tube having a distal end for insertion into a patient's rectum and a proximal end for inserting an obturator and an insertion stop adapted over the main tube for inserting the speculum into the patient's rectum at a predetermined depth.

According to the fifth embodiment of the present invention, the insertion stop is adapted removably over the main tube.

According to the sixth embodiment, the speculum includes an adjustment means for adjusting the insertion stop to a length of the speculum in rectum to insert the speculum at predetermined depth.

According to the present invention, the adjustment means includes a locking tab on the main tube and a locking means adapted on the insertion stop for adjusting the insertion stop to the predetermined depth over the main tube.

In seventh embodiment, the present invention provides a washer cone made of soft gel material for snugly fitting of the speculum over the rectal area of the patient and providing comfort to the patient and prevent leaking of waste fluid material between speculum and anal wall.

### BRIEF DESCRPTION OF THE DRAWINGS

The accompanying drawings, which are incorporated into and form a part of the specification, schematically illustrate one or more exemplary embodiments of the present invention and together with the general description given above and detailed description of the preferred embodiments given below serve to explain the principles of the invention:

FIG. 1 illustrates a sectional view of a speculum assembly with connections according to the present invention;

FIG. 2 illustrates an exploded view of the speculum kit according to the present invention; and

FIG. 3 illustrates a perspective view of the combo system according to the present invention.

### DETAILED DESCRIPTION OF THE PRESENT INVENTION

The present invention provides a speculum for performing colonoscopy in combination with colon irrigation/cleansing, a compact system comprising a colon irrigation device and a colonoscopy device, and a method for performing colonoscopy along with colon irrigation/cleansing. The present invention also provides a disposable kit with the speculum and a washer cone.

In general, the speculum according to the present invention comprises a main tube having a proximal end and distal end, and an inlet tube and an outlet tube intersecting to the main tube.

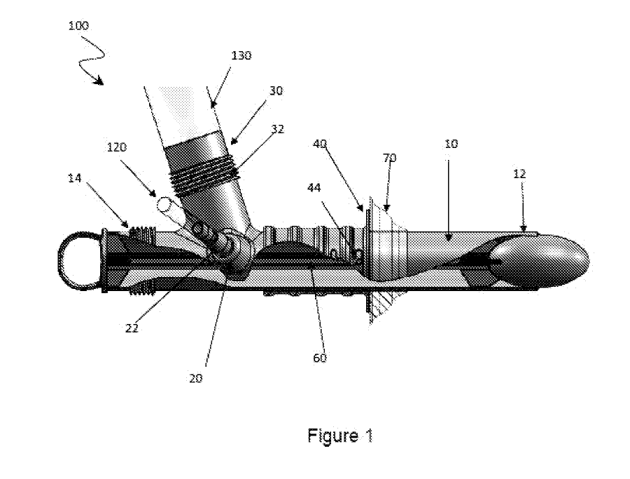

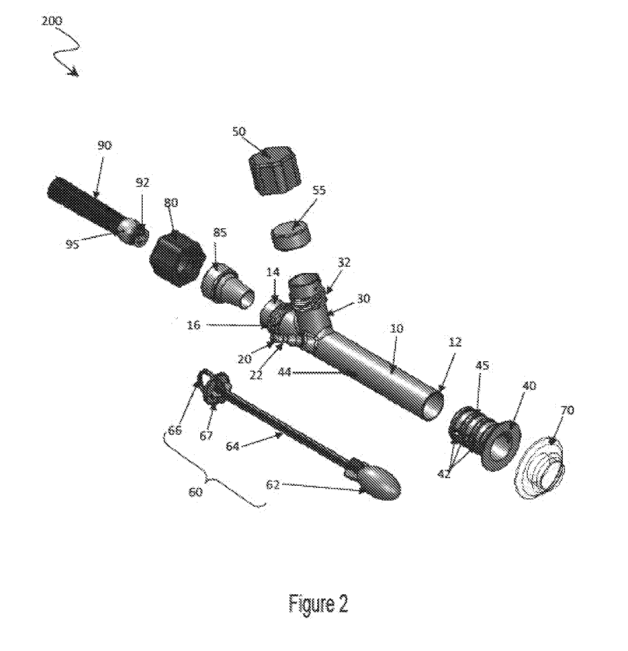

Referring FIG. 1 shows an assembled speculum (100) according to the present invention with the connections for carrying out colon irrigation and FIG. 2 shows a speculum kit (200) and/or an exploded view of the speculum according to the present invention.

The speculum kit (200) comprises a rectal speculum (100), an obturator (60) for facilitating insertion of the rectal speculum (100) in a patient's rectum (not shown) and a penetration cap (80) with a seal skirt (85) for inserting a colonoscope (90).

The rectal speculum (100) as shown in FIGS. 1 and 2 comprises an inlet tube (20) for injecting irrigation fluid and an outlet tube (30) for waste removal intersecting to an unobstructed main tube (10) and a cap (50) for carrying out colon irrigation with a washer (55) to avoid leakage.

The main tube (10) includes a proximal end (14) for inserting the obturator (60) or a colonoscope (90) and a distal end (12) adapted for insertion in a patient's rectum. Preferably, the main tube is cylindrical. Alternatively, the main tube may be tapering towards its distal end.

The inlet tube (20) intersects near the proximal end (14) of the main tube (10) at an obtuse angle with respect to the distal end (12) of the main tube (10) as shown in FIGS. 1 and 2 for directing irrigation fluid into the said main tube (10). According to the present invention the inlet tube has a diameter less than the diameter of main tube. The inlet tube (20) has annular beads (22) as shown in FIGS. 1 and 2 for push on or snap fitting of a hose (120) of treatment fluid of the colon irrigation device.

The outlet tube (30) is tapered for snug press fit of the waste discharge hose (130), and intersects the said main tube (10) at an obtuse angle with respect to the distal end (12) of the main tube (10) and away from the distal end is (12) for connecting to a waste discharge hose (130) of the irrigation device.

According to the present invention, the outlet tube has a diameter substantially equal to the diameter of the said main tube. Alternatively, the diameter of the outlet tube can be different depending upon the diameter of an inlet of the view glass of the colon irrigation device. The outlet tube (30) has threads (32) as shown in FIGS. 1 and 2 for fitting the waste hose of the irrigation device. Alternatively, other known snug fittings can be used for the outlet tube.

According to the present invention, the inlet tube (20) and the outlet tube (30) intersect preferably adjacent to the main tube (10). Alternatively, the inlet tube and the outlet tube are intersects can be away from each other to the main tube.

According to the present invention, the inlet tube (20) and the outlet tube (30) are intersects preferably at the same distance from the distal end of main tube (10). Alternatively, the inlet tube and the outlet tube can intersect at different distance from the distal end of the main tube.

The speculum (100) also comprises an insertion stop (40) adapted removably over the main tube (10). An adjustment means (42, 44) is provided for adjusting the insertion length of the main tube (10) of the speculum at predetermined depth. The adjustment means has a locking tab (44) on the main tube (10) and a locking means (42,45) adapted on the insertion stop (40) for adjusting the insertion stop (40) to a length equal over the main tube (10) to the predetermined depth required to insert in the patient's rectum as shown in FIG. 1.

To provide comfort and avoid pain and prevent leaking of irrigation fluid around the speculum during the insertion of the speculum as well as during the colon irrigation/cleansing and/or colonoscopy process, the present invention provides a washer cone (70) as shown in FIGS. 1 and 2 adapted in front of the insertion stop (40). The washer cone (70) is made of soft gel material such as silicone, rubber, air balloon, or other suitable materials.

The obturator (60) used in combination with the speculum (200) comprises an elongated rod (64) having a known round or elongated shape tip portion (62) at the distal end (12) for insertion in the patient's rectum and a handle (66) and an obturator insertion stop (67) at the proximal end (14) of the main tube (10) of the speculum. As shown in FIG. 1, the obturator (60) is inserted from the proximal end (14) of the main tube (10) of the speculum (100) in order to facilitate insertion of the speculum (100) in the patient's rectum (not shown).

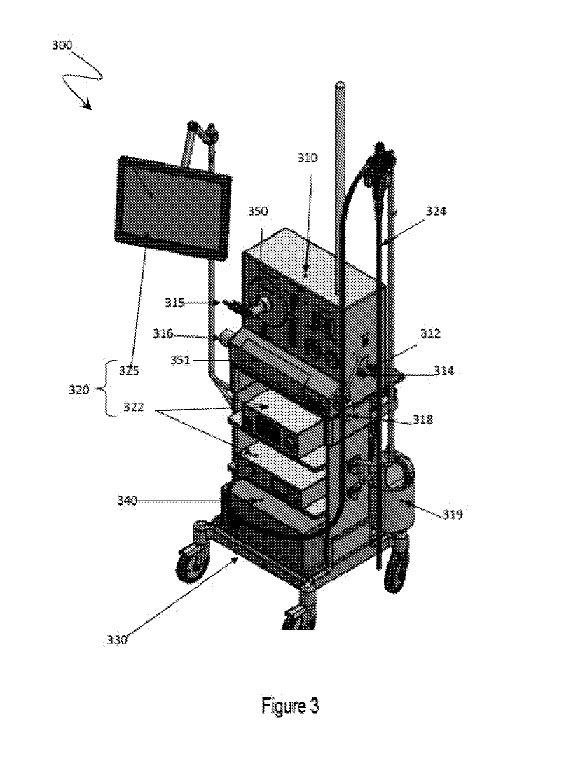

Referring FIG. 3 shows a system (300) for carrying out colonoscopy in combination with the colon irrigation/cleansing. The system (300) comprises a standard colon irrigation device (310), a standard colonoscopy device (320) and an integrated power supply device (340) for both devices. Alternatively, the devices can be separately used for carrying out the colonoscopy in combination with the colon irrigation/cleansing with separate power connections. The system as shown in the FIG. 3 is adapted over a trolley (330). The colon irrigation device (310) comprises known elements such as a hot and cold water connections (312, 314), a disinfecting solution (319), a water mixer (not shown) with colonoscopy in combination with the colon irrigation/cleansing temperature adjustment (350), pressure and flow controllers (not shown), pressure and temperature gauges (not shown), a waste fluid inlet (316) and outlet (318), a waste viewing window (351) and a speculum of the present invention. The colonoscopy device (320) comprises known components such as a colonoscope (324), a colon imaging display (325), and other colonoscopy known device components (322). A colon irrigation device of one of the inventors of this application as disclosed in the U.S. patent application Ser. Nos. 13/228209 and 13/235225 can be used for colon irrigation/cleansing.

The present invention also provides a method for carrying out colonoscopy in combination with the colon irrigation/cleansing. To carry out colon irrigation/cleansing, the treatment fluid hose of the colon irrigation device is connected to the outlet port of the colon irrigation device and is fixed over the inlet tube of the speculum and the waste hose of the colon irrigation device is connected with the waste fluid inlet and with the outlet tube of the speculum. Then a rectal examination is carried out and the insertion stop is adjusted over the tab provided on the main tube of the speculum. The washer cone is then inserted over the main tube. Then the obturator inserted from the proximal end through the hollow interior to extend through the distal end of the main tube of the speculum. Now, the speculum is ready for insertion in the patient's rectum as shown in FIG. 1. The speculum is then inserted in the patient's rectum with slight twisting. The insertion stop helps to insert the distal end of the speculum at predetermined depth and the washer cone not only provides a conform level to the patient but also acts as a seal to prevent leaking. Once the speculum has been properly positioned, the obturator is then removed from the speculum by use of the handle, and the cap with the washer is removably fixed over the proximal end of the speculum thus closing the proximal end of the rectal speculum. Then colon irrigation is carried out with the known ways that is by filling the colon with colon irrigation fluid or water and then emptying the colon repeatedly. This filling and emptying cycle is carried out as per the patients individual requirement or for predetermined numbers. Meanwhile, the colonoscope is made ready for colonoscopy by inserting it through the penetration cap and the seal skirt and attaching the buoyancy cuff at the tip of colonoscope. After emptying the final cycle as decided, the cap is removed from the proximal end of the main tube of the speculum and the colonoscope is inserted through the proximal end of the main tube of the speculum and the colon is filled slowly with the irrigation fluid or water and advancing the colonoscope up to secum of the colon. The buoyancy cuff helps to float the colonoscope tip in the irrigation fluid or water in the colon for easier navigation of the scope to reach the intended target in the intestinal tract. This can be aided by gently pumping the waste hose by hand creating a wave action in the colon. Once the colonoscope reaches the intended target, the colon is emptied while the colonoscope is held in place and the colonoscopy screening of the colon is carried out. The speculum may be removed from the patient's rectum and allow to remain over the outer casing of the colonoscope until the screening examination is completed.

The speculum of the present invention allows carrying out colonoscopy screening immediately following the colon irrigation which saves time. In the existing speculum designs, the waste water hose of the colon irrigation device is connected to the proximal end of the main tube after the insertion of the speculum in the patient's colon, which may results in slip of the inserted speculum and also pains to the patient, moreover, it may hurt the patient. In the present invention, the hoses of irrigation fluid and waste are connected before the insertion of the speculum in the patient's rectum and hence it avoids the connections of the hoses after the insertion.

If by advancing the colonoscope it is determined that the colon is not sufficiently clean, the scope can be drawn back and the irrigation/cleansing of the colon can then be repeated by reattaching the cap to the proximal end of the main tube, followed by repeating of the insertion of the colonoscope for re-screening of the colon. This avoids not only incomplete or failed screenings but also the need for re-scheduling of colonoscopy for the patients to repeat the procedures.

By using floating and wave action while advancing the colonoscope along the colon, less pressure is exerted in the present invention on the colon wall resulting in less injuries or perforations of the colon normally associated presently used techniques with conventional colonoscopy procedures.

By using irrigation fluids or water to clean the colon, the laxative preparations normally used as conventional colonoscopy preparation are reduced or eliminated in the present invention resulting in better toleration by the patients without or less of the side effects that are normally associated with conventional laxative preparations.

By using irrigation fluids or water to advance the colonoscope along the colon, less pressure is exerted on the colon wall resulting in less pain that is normally associated with conventional colonoscopy procedures by virtue of the strong pushing force used to advance the colonoscope. As a result the need for anesthesia is reduced or eliminated resulting in reduced treatment time since the post procedure recovery time of the patients is reduced or eliminated.

The foregoing is illustrative of the present invention and is not to be construed as limiting thereof. The electric connection and other known parts are not described herein in detail as the person skilled in the art can easily implement those things in the present invention, Further though a few exemplary embodiments of the present invention have been described, those skilled in the art will readily appreciate that many modifications are possible without materially departing from the novel teachings and advantages of the present invention. Accordingly, all such modifications are intended to be included within the scope of the present invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein:

1. A rectal speculum for carrying out the colonoscopy in combination with colon irrigation/cleansing, said speculum comprising:
a main tube having a distal end for insertion into a patient's rectum and a proximal end for inserting an obturator or a colonoscope;
an inlet tube for directing irrigation fluid into the said main tube intersecting near the proximal end of the main tube;
an outlet tube for connecting to a waste discharge hose intersecting near the proximal end of the main tube; and
a cap for closing the proximal end of the main tube for carrying out the irrigation/cleansing.

2. The rectal speculum of claim 1, wherein said inlet tube having a diameter less than the diameter of main tube.

3. The rectal speculum of claim 1, wherein said outlet tube having a diameter substantially equal to the diameter of the said main tube.

4. The rectal speculum of claim 1, wherein said inlet tube and outlet tube intersect the said main tube at an obtuse angle with respect to the distal end and away from the distal end.

5. The rectal speculum of claim 1, wherein the inlet tube and the outlet tube are intersecting preferably adjacent to the main tube.

6. The rectal speculum of claim 1 wherein the inlet tube and the outlet tube are intersecting preferably at the same distance from the distal end of main tube.

7. The rectal speculum of claim 1 wherein the inlet tube and the outlet tube can intersect at different distance from the distal end of the main tube.

8. The rectal speculum of claim 1 further comprises an insertion stop adapted removably over the main tube.

9. The rectal speculum of claim 8 further comprises an adjustment means for adjusting the insertion stop to a position on the main tube of the speculum for insertion of the distal end into the rectum at a predetermined depth.

10. The rectal speculum of claim 9, wherein the adjustment means includes a locking tab on the main tube and a locking means adapted on the insertion stop for adjusting the insertion stop to the predetermined depth over the main tube.

11. The rectal speculum of claim 1, which includes a washer cone made of soft gel material adapted for snugly fitting of the speculum over the rectal area of the patient and providing comfort to the patient and prevents leaking of treatment fluid and waste fluid material.

12. The rectal speculum of claim 1, wherein said main tube is cylindrical.

13. The rectal speculum of claim 1, wherein a distal part of the main tube can be tapered toward the said distal end.

14. A rectal speculum kit for carrying out colonoscopy in combination with colon irrigation/cleansing, said speculum assembly comprising:
a rectal speculum, said speculum including:
a main tube having a distal end for insertion into a patient's rectum and a proximal end for inserting a colonoscope;
an inlet tube for directing fluids into the said main tube, said inlet tube having a diameter less than the diameter of main tube and intersecting near the proximal end of the main tube at an obtuse angle with respect to the distal end of the main tube;
an outlet tube for connecting to a waste discharge hose, said outlet tube having a diameter substantially equal to the diameter of the said main tube and intersecting the said main tube at an obtuse angle with respect to the distal end and away from the distal end;
a cap for closing a proximal end of the main tube for carrying out irrigation/cleansing;
an obturator removably positioned in the main tube for facilitating insertion of speculum in a patient's rectum, said obturator is inserted from the proximal end of the main tube; and
a penetration cap with a seal skirt for inserting a colonoscope from the proximal end of the main tube.

15. A rectal speculum kit of claim 14, wherein the kit also comprises a buoyancycuff to adapt to the tip of the colonoscope.

16. A rectal speculum kit of claim 14, wherein the kit is a disposable or single use kit.

17. A system for carrying out colonoscopy in combination with colon irrigation/cleansing, said system comprising:
a colon irrigation equipment for carrying out irrigation/cleansing of the colon;
a colonoscopy equipment for carrying out colonoscopy or colon screening including a colonoscope and a buoyancy cuff to adapt at the tip of the colonoscope;
a rectal speculum, said speculum including:
a main tube having a distal end for insertion into a patient's rectum and a proximal end for inserting the colonoscope with the buoyancy cuff;
an inlet tube for directing fluids into the said main tube, said inlet tube having a diameter less than the diameter of main tube and intersecting near the proximal end of the main tube at an obtuse angle with respect to the distal end of the main tube wherein said inlet tube is connected to the treatment fluid outlet port of the colon irrigation equipment;
an outlet tube for connecting to a waste discharge hose, said outlet tube having a diameter substantially equal to the diameter of the said main tube and intersecting the said main tube at an obtuse angle with respect to the distal end and away from the distal end wherein the outlet tube is connected to discharge port of the colon irrigation equipment;
a cap for closing a proximal end of the main tube for carrying irrigation;
an obturator removably positioned in the main tube for facilitating insertion of speculum in a patient's rectum, said obturator is inserted from the proximal end of the main tube; and
a penetration cap with a seal skirt for inserting a colonoscope from the proximal end of the main tube.

18. A method for carrying out colonoscopy in combination with colon irrigation/cleansing, comprising steps of:
fitting a fluid hose to the inlet tube for receiving irrigation fluid and waste removal hose to the outlet tube of the speculum;
inserting obturator from proximal end of the main tube;
inserting the speculum into rectum with slight twisting at a predetermined depth;
removing obturator from the speculum;
closing the proximal end of the main tube with a cap;
carrying out irrigation of colon with repeated fill and empty cycles;
removing the cap from the proximal end of the main tube after a predetermined last empty cycle of irrigation;
inserting the tip of the colonoscope through the penetration cap and the seal skirt;
attaching the buoyancy cuff to the tip of colonoscope;
inserting the colonoscope into the main tube at proximal end;
fitting the penetration cap on the proximal end of the speculum;
filling the colon slowly with the treatment fluid or water and advancing the colonoscope up to secum of the colon; and
emptying the colon of the treatment fluid or water and carrying out the colonoscopy.

19. A method of claim 18, wherein the step of inserting the speculum into rectum with slight twisting at a predetermined depth in a patient's rectum includes a step of examining the patient's rectum for determining depth for inserting speculum and adjusting an insertion stop to a length on the speculum equal to the predetermined depth by engaging the insertion stop with locking tab.

20. A method of claim 18, wherein the step of advancing the colonoscope up to the secum or intended target of the intestinal tract includes allowing floating of the colonoscope along with steering of the colonoscope and pulsating wave action of the fluid in the colon.

21. A method of claim 18, wherein inserting the speculum through a washer cone made of soft gel material for snugly fitting of the speculum over the rectal area of the patient and providing comfort to the patient and prevents leaking of treatment fluid and waste fluid material.

22. A method of claim 18 further includes repeating the colon irrigation/cleansing process by removing colonoscope and fitting cap to the proximal end of the speculum if colon is not cleaned properly and thereafter repeating the steps of the colonoscopy.

23. A rectal speculum comprising a main tube having a distal end for insertion into a patient's rectum and a proximal end for inserting an obturator and an insertion stop adapted over the main tube for inserting the speculum into the patient's rectum at a predetermined depth.

24. The rectal speculum of claim 23, wherein the insertion stop is adapted removably over the main tube.

25. The rectal speculum of claim 23, which includes an adjustment means for adjusting the insertion stop to a length of the speculum in rectum to insert the speculum at predetermined depth.

26. The rectal speculum of claim 23, wherein the adjustment means includes a locking tab on the main tube and a locking means adapted on the insertion stop for adjusting the insertion stop to the predetermined depth over the main tube.

27. A washer cone made of soft gel material for snugly fitting a speculum over the rectal area of the patient and providing comfort to the patient and prevents leaking of waste fluid material between speculum and anal wall.

28. The washer cone of claim 27, wherein soft gel material includes silicone, rubber, air balloon, or other suitable materials.
